(19) United States
(12) Patent Application Publication
(10) Pub. No.: US 2002/0028765 A1
(43) Pub. Date: Mar. 7, 2002

(54) Process for improving the growth of human head hair

(76) Inventor: Armin Maurer, Korb (DE)

Correspondence Address: GREENBLUM & BERNSTEIN, P.L.C., 1941 ROLAND CLARKE PLACE, RESTON, VA, 20191, US

(21) Appl. No.: 09/944,414

(22) Filed: Sep. 4, 2001

(30) Foreign Application Priority Data
Sep. 5, 2000 (DE) 100 43 982.9

(51) Int. Cl. A01N 37/18; A61K 38/00; A61K 7/06; A61K 7/11; A61K 39/00; C07K 1/00; C07K 14/00

(52) U.S. Cl. 514/2; 514/12; 530/350; 424/70.14; 424/185.1

(57) ABSTRACT

Process for improving growth of human head hair, wherein a substance containing botulinum toxin is injected, such as by subcutaneous or intramuscular injection, in those parts of the head in which the circulation of blood or supply of nutrients to the follicles which are essential for hair growth, are disturbed.

### BACKGROUND OF THE INVENTION

1. Field of the Invention

The invention relates to a process for improving the growth of human head hair.

2. Discussion of Background Information

Under the scalp for human head hair, the root of each individual hair is embedded in a pocket-shaped structure, the hair follicle. At the end of this hair follicle there are tiny capillary blood vessels which provide nourishment to the hair root. The skull is covered with a web of blood vessels resembling the branches of a tree. These supply the capillary blood vessels of the hair follicles with blood and nutrients. The skull is also covered by flat sinew and muscular cords.

According to the current state of the art, medicaments (for internal and external application) and (mechanical and psychosomatic) methods are known, with the assistance of which the loss of head hair can be stopped or new growth stimulated.

A number of these medicaments contain anti-androgens which reduce the production of male hormones (androgens). Genes and hormones play the most significant part in so-called androgenetic hair loss.

This type of hair loss is preceded by changes or hardening in the connective tissue at the base of the hair. The hair is no longer held firmly or adequately nourished.

If a hair follicle with a predisposition to baldness is exposed to male hormones over a longer period of time, it produces fewer hairs.

Some anti-androgen medicaments cut off the access of androgens to the hair root. These are applied to the scalp. Other anti-androgen medicaments inhibit the transformation of testosterone (male sexual hormone) into dihydrotestosterone (DHT), which causes congenital baldness. These are taken orally.

The effectiveness of many of these medicaments is questionable and their side effects often lead to reductions in dosage. No account is taken of the fact that, with many of those affected, hair loss takes place only in certain areas of the scalp.

According to prior art, hair transplantations are also known, where existing healthy hair is transplanted to areas of impaired hair growth in the same person.

Numerous patents are known whose purpose is the prevention of hair loss:

According to German Patent No. 32 28 489 goat's milk butter, testes and placenta extracts have a slight but constant effect in counteracting hair loss.

U.S. patent application Ser. No. 08/994,347, filed Dec. 19, 1997, which matured as U.S. Pat. No. 6,030,948, describes hair regeneration compositions for treatment of congenital baldness (alopecia) and method of application related thereto.

U.S. Pat. No. 6,020,327 describes a process for the treatment of hair loss where an aromatase inhibitor is administered.

U.S. Pat. Nos. 5,538,945 and 6,017,888 describe the stimulation of hair growth by a peptide-copper complex.

U.S. Pat. No. 5,861,142 describes a process for encouraging the keratinization of hair, nails and skin by the therapeutic administration of benzimidazoles.

Many of the above substances and methods have produced only very slight or questionable effect. Therefore, there is still a need to according to the present invention to describe a process for improving the growth of human head hair which has a high probability of success, in particular for a selected group of persons.

### SUMMARY OF THE INVENTION

The present invention relates to processes for improving growth of human head hair.

The present includes processes for improving growth of human head hair, comprising injecting a substance containing botulinum toxin into at least one portion of the head in which at least one of circulation of blood and supply of nutrients to the follicles which are essential for hair growth are disturbed.

The injecting a substance can comprise at least one of subcutaneous and intramuscular injection.

The botulinum toxin can be botulinum toxin type A.

The substance that is injected can comprise sodium chloride solution as a basic constituent.

The injecting can be repeated, such as wherein the repeating the injecting comprises repetition of the injecting afer, at the earliest, 10 weeks.

The process can further include tensing of muscles around a site of injecting subsequent to injecting.

### DETAILED DESCRIPTION OF THE PRESENT INVENTION

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show structural details of the present invention in more detail than is necessary for the fundamental understanding of the present invention, the description making apparent to those skilled in the art how the several forms of the present invention may be embodied in practice.

The process of the present invention makes use of the antispasmodic characteristics of botulinum toxin. Botulinum toxin is a neurotoxin produced by the anaerobic bacterium Clostridium Botulinum. There are eight serologically distinct botulinum toxins, of which type A is the most widely studied. The use of botulinum toxin for various medical indications is known in the prior art. However, these applications do not pertain to the use of this toxin for improving the growth of human head hair.

In therapeutic applications, tiny quantities of botulinum toxin type A are injected directly into selected muscles to counteract involuntary contractions.

When botulinum toxin is injected into a muscle, certain nerve impulses are blocked, thereby reducing the tonus of the muscle in question. The full effect is achieved only after a delay of several days. The therapeutic objective of local muscular relaxation with botulinum toxin is dependent on the individual symptoms and the requirements of the patient. The temporary muscular paralysis caused by botulinum toxin is temporary and reversible.

The following are the most common applications for botulinum toxin:

Treatment of eyelid spasm (idiopathic blepharospasm),

Hemifacial spasm (coexisting hemifacial dystonic movement),

Spasmodic torticollis (idiopathic rotating torticollis).

Equinovelgus deformity.

In cosmetic medicine, botulinum toxin A is used, among other things, for the treatment of forehead wrinkles and crow's feet. Clinical studies suggest that it may also be useful in treating writer's cramp, etc. Botulinum toxin type A is most commonly available in dry solid form. Other constituents of the dry solid may be human albumin, lactose or sodium chloride. Before administering the injection, the dry solid is dissolved in a sterile physiological sodium chloride solution.

Specific indications for botulinum toxin are described in the following patents, each of which is incorporated by reference herein in its entirety:

U.S. Pat. No. 5,766,605 describes a method of treatment for autonomic nerve dysfunction with botulinum toxin;

U.S. Pat. No. 5,714,468 describes a process for the reduction of migraine headache pain using botulinum toxin;

U.S. Pat. No. 5,053,005 describes a non-surgical method of modulating spinal curvature in developing vertebrates using botulinum toxin;

U.S. Pat. No. 4,932,936 describes method and apparatus for pharmacological control of spastic urethral sphincters by injections of botulinum toxin A;

German Patent DE 43 35 366 describes a process for manufacturing a crystalline botulinum toxin type A as a therapeutic medication for squints and blepharospasm;

U.S. Pat. No. 5,721,215 describes an injectable therapy for control of muscle spasms and pain related to muscle spasms using botulinum toxin;

U.S. Pat. No. 5,731,161 describes a botulinum toxin antibody-detection assay; and

U.S. Pat. No. 5,939,070 describes a hybrid botulinal neurotoxin.

In all the above applications of botulinum toxin, it is used primarily for the treatment of involuntary muscular spasms. Generally speaking, the effects of botulinum toxin do not become noticeable until after about 3-4 days. The duration of the therapeutic effect is between 3 and 10 months. A botulinum toxin treatment can be repeated indefinitely, however, normally not until 10 weeks have expired.

The invention is based on the finding that hair-growth disorders in a certain group of people are attributable to a disruption in the supply of blood and nutrients to the hair follicles caused by stress.

It is known (e.g., from the source “Aroma Therapy for Common Ailments”, Gaia Books, London 1991) that sporadic hair loss may be part of a reaction to severe stress or shock. “Stress in particular may lead to tension in the scalp, thereby preventing blood and nutrients from reaching the individual hair follicles. The starving hair roots then shrink in the follicle and the hairs drop out”.

This group of persons, whose typical reaction to stress involves cramps and tension in certain areas of the head (skin, muscles-e.g., by gritting teeth or clenching jaws) are particularly susceptible to hair-growth problems. Among the parts of the head which may be affected are those which are essential for the growth of head hair, i.e., for the supply of blood and nutrients to the hair follicles.

It should be emphasized that these areas of the head include the scalp and hair as well as the muscles by which the circulation of blood in the scalp may be disrupted (e.g., through tension of the musculus temporalis with the resulting reduction in the quantity of blood passing through this muscle to supply the hair follicles, or through the competing demand for blood and nutrients on the part of the permanently tensed muscle). The resulting loss of hair is most commonly seen in the front area of the head and temples, causing the hairline to recede.

This is due to the anatomical position of the blood vessels, muscles and sinews in the scalp. In order to reduce these tensions and improve the growth of hair on the human scalp, according to the invention, injections of a substance containing botulinum toxin are administered to the areas of the head in which the circulation of blood, essential for the supply of nutrients to the hair follicles and consequent hair growth, is disturbed.

According to the invention, these injections may be either subcutaneous or intramuscular. Muscular tension, in particular of the musculus temporalis, which runs across the scalp on both sides, can be measured more accurately by means of muscle-tonus measurements (electromyographic measurements, EMG). In this way, the injections can be precisely placed, thereby increasing the success of botulinum toxin therapy in improving the growth of hair.

To optimize the absorption of the botulinum toxin by the contracted muscles and increase its paralytic effect, these muscles should preferably be consciously tensed several times every 15 minutes for a period of two hours following the injection. During the administration of the injection and for several hours thereafter, the patient should preferably remain in an upright position. After administration, the injected botulinum toxin diffuses into the tissue adjacent to the point of injection. It should be noted that the administration of botulinum toxin according to the invention should be carried out by qualified doctors only.

The group of persons reacting to stress with muscular tension in the head area is easily defined. In many cases, tension of this kind is visible (formation of wrinkles), tangible or detectable by EMG (electromyographic measurement). With the group of persons concerned, it is occupational stress which manifests itself particularly through tension and consequent loss of hair in these areas of the head. It is this group of persons at which the process for improving hair growth according to the invention is aimed.

Of all the botulinum toxins, it is type A whose effects have been most widely studied. It is therefore type A which is preferred for the process according to the invention.

In the process according to the invention, the botulinum toxin is injected in the same way as with previously known indications using a physiological sodium chloride solution. The sustained success of the therapy is assured by repeating the injections at intervals of at least I0 weeks, or more usually, of several months.

It is noted that the foregoing examples have been provided merely for the purpose of explanation and are in no way to be construed as limiting of the present invention. While the present invention has been described with reference to an exemplary embodiment, it is understood that the words which have been used herein are words of description and illustration, rather than words of limitation. Changes may be made, within the purview of the appended claims, as presently stated and as amended, without departing from the scope and spirit of the present invention in its aspects. Although the present invention has been described herein with reference to particular means, materials and embodiments, the present invention is not intended to be limited to the particulars disclosed herein; rather, the present invention extends to all functionally equivalent structures, methods and uses, such as are within the scope of the appended claims.

I claim:

1. Process for improving growth of human head hair, comprising:
injecting a substance containing botulinum toxin into at least one portion of the head in which at least one of circulation of blood and supply of nutrients to the follicles which are essential for hair growth are disturbed.

2. The process according to claim 1 wherein the injecting a substance comprises at least one of subcutaneous and intramuscular injection.

3. The process according to claim 2 wherein the injecting a substance comprises subcutaneous injection.

4. The process according to claim 3 wherein the injecting a substance comprises intramuscular injection.

5. The process according to claim 1 wherein the botulinum toxin is botulinum toxin type A.

6. The process according to claim 1 wherein the substance comprises sodium chloride solution as a basic constituent.

7. The process according to claim 1 comprising repeating the injecting.

8. The process according to claim 7 where the repeating the injecting comprises repetition of the injecting after, at the earliest, 10 weeks.

9. The process according to claim 1 comprising tensing of muscles around a site of injecting subsequent to injecting.
